(19) United States
(12) Patent Application Publication
(10) Pub. No.: US 2026/0242778 A1
(43) Pub. Date: Aug. 20, 2026

(54) SYNTHETIC MRNA ENCODING HIF-1ALPHA AND USES FOR WOUND HEALING

(71) Applicant: The Johns Hopkins University, Baltimore, MD (US)

(72) Inventors: John Martin ABRAHAM, Woodbine, MD (US); John W. HARMON, Baltimore, MD (US); Jakub WLODARCZYK, Baltimore, MD (US); Emma Katherine PIPPENGER, Glenwood, MD (US); Albert LENG, Palo Alto, CA (US); Sanaz NOURMOHAMMADI ABADCGI, Baltimore, MD (US); Niloufar SHABABI, Baltimore, MD (US); Mohsen ROUHANI RAVARI, Baltimore, MD (US); Jinny HA, Glenwood, MD (US)

(73) Assignee: The Johns Hopkins University, Baltimore, MD (US)

(21) Appl. No.: 19/472,482

(22) PCT Filed: Apr. 5, 2024
(86) PCT No.: PCT/US2024/023344

§ 371 Date: Oct. 3, 2025

Related U.S. Application Data
(60) Provisional application No. 63/494,434, filed on Apr. 5, 2023.

(51) Int. Cl. C12N 15/11; A61K 31/7105; A61K 38/17; A61K 48/00; C07K 14/47; C12N 9/12; C12P 19/34

(52) U.S. Cl. CPC C12N 15/11; A61K 31/7105; A61K 38/17; A61K 48/005; C07K 14/4702; C12N 9/1247; C12P 19/34; C12Y 207/07006

(57) ABSTRACT

The present embodiment relates to a synthetic HIF-1a mRNA comprising an open reading frame encoding at least one of a polypeptide derived from a HIF-1a protein or a fragment thereof, wherein the polypeptide or fragment thereof elicits the expression of at least one of an angiogenic factor such as VEGF, PGF and angiotensin involved in wound healing. The present embodiment also relates to the therapeutic uses of synthetic HIF-1a mRNA or method for treating a surgical wound or preventing ischemia or necrosis in wound tissue, reconstituted with a pedicle flap.

### RELATED APPLICATION

This application claims priority to U.S. Provisional Application No. U.S. 63/494,434, filed Apr. 5, 2023, the content of which are incorporated herein by reference in their entirety.

### SEQUENCE LISTING

The instant application contains a Sequence Listing XML which has been submitted electronically and is hereby incorporated by reference in its entirety. Said XML copy, created on Mar. 27, 2023, is named 002240_589508 and is 93,296 bytes in size.

### BACKGROUND

### 1. Technical Field

Currently claimed embodiments of the invention relate to synthetic mRNAs encoding the hypoxia-inducible factor-1a (HIF-1α), methods for producing the synthetic HIF-1α mRNAs and their applications in wound healing.

### 2. Discussion of Related Art

Wounds pose significant threat to public health and the economy due to the increasing prevalence of diabetes, obesity, and aging population. In the US, annual costs for wounds management are staggering at over $25 billion for the care of more than 6.5 million patients. Major wounds are often treated using surgical debridement. Wound reconstruction carried out with pedicle flaps can lead to ischemia or necrosis. Amputations of lower extremities are examples. The goal is to achieve a minor amputation leading to successful rehabilitation. If the minor procedure fails, and higher amputation is required, the chance of successful rehabilitation greatly diminishes. From clinical experience, vascular perfusion of the distal portions of the flap is the critical determinant of flap survival leading to the successful reconstruction of the limb.

Patients whose lower limb minor amputations fail because of flap necrosis, have a failure rate reported at 35%. Flap failure is also a significant risk for patients undergoing abdominoplasty after bariatric surgery, advanced ventral hernia repair, reconstruction after civilian or military trauma, and many other conditions. To overcome ischemia, surgical delay to precondition flaps is achieved in a variety of methods. However, the methods share the common feature of producing temporary ischemia in the flap leading to angiogenesis and other mechanisms of survival which can be instituted endogenously prior to mobilizing the flap for the intended purpose of reconstruction. The flap is raised to induce a moderate amount of ischemia. An example of this strategy is to raise a bilateral pedicle flap attached at both ends to provide two sources of blood supply. For the reconstruction, it is converted into a unilateral flap. Clearly, a simple medical alternative would be superior to the surgical strategies currently in use.

Systemic treatment with calcium channel blockers has been assessed extensively in animal studies with modest benefits.

Localized delivery of HIF-1α DNA plasmid showed potential clinical benefit in wound healing. However, a DNA expression system requires delivery to the nucleus where the plasmid may be integrated, thus resulting in genomic alteration. In addition, a DNA expression plasmid usually constitutively expresses the encoded gene indefinitely which can lead to negative consequences.

The search for an effective and practical method to promote the viability of surgical pedicle flaps has been a focus of research for decades. During the wound healing process of pedicle flaps, severe complications due to ischemia or necrosis may occur. Therefore, there is an unmet need to develop a therapeutic compound that sustains the viability of surgical pedicle flaps for faster and more efficient wound healing following surgery.

### SUMMARY OF THE INVENTION

An embodiment relates to a synthetic HIF-1α mRNA that includes an open reading frame encoding at least one of a polypeptide derived from a HIF-1α protein or a fragment thereof. The polypeptide or fragment thereof elicits the expression of at least one of an angiogenic factor involved in wound healing.

The synthetic HIF-1α mRNA of the preceding embodiment, wherein the wound is caused by a surgical procedure and is reconstructed with a pedicle flap.

The synthetic HIF-1α mRNA of any preceding embodiments, wherein the open reading frame comprises at least one of a mutation encoding for an amino acid residue susceptible to hydroxylation under conditions of normoxia.

The synthetic HIF-1α mRNA of the preceding embodiment, wherein the amino acid residue is a proline.

The synthetic HIF-1α mRNA of the preceding embodiment, wherein the amino acid residue is the proline residue at position 564 and/or the proline residue at position 402, and wherein the numbering of the amino acid residues is that of the human HIF-1α protein.

The synthetic HIF-1α mRNA of any preceding embodiments, wherein the open reading frame comprises at least one of a nucleic acid sequence having at least 90%, at least 95% or 100% identity with the nucleic acid sequence SEQ ID NO: 17 and a fragment thereof.

The synthetic HIF-1α mRNA of any preceding embodiments, wherein the open reading frame comprises at least one of a nucleic acid sequence having at least 90%, at least 95% or 100% identity with the nucleic acid sequence SEQ ID NO: 18 and a fragment thereof.

The synthetic HIF-1α mRNA of any preceding embodiments, wherein the open reading frame comprises at least one of a nucleic acid sequence having at least 90%, at least 95% or 100% identity with the nucleic acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and ID NO: 24, and a fragment thereof.

The synthetic HIF-1α mRNA of any preceding embodiments, further comprising at least one of a poly-A tail, a methylated cap, a 5′ untranslated region (5′-UTR), a 3′ untranslated region (3′-UTR) or any combination thereof.

The synthetic HIF-1α mRNA of any preceding embodiments, wherein at least one of a nucleobase of the nucleic acid sequence is modified by methylation, isomerization or both.

The synthetic HIF-1α mRNA of any preceding embodiments, wherein the at least one of the nucleobase is selected from the group consisting of pseudouridine (PsU), N1-methyl-pseudouridine (1mPsU), 5-methyl-uridine (5mU), 2′-O-methyl-uridine (2′-O-methyl-U), 5-methyl-cytidine (5mC), 2-thiouridine (s2U), N6-methyladenosine (6 mA), and any combination thereof.

The synthetic HIF-1α mRNA of any preceding embodiments, wherein the at least one of the nucleobase is N1-methyl-pseudouridine (1mPsU).

The synthetic HIF-1α mRNA of any preceding embodiments, wherein the at least one of the angiogenic factors is selected from the group of consisting of vascular endothelial growth factor (VEGF), placental growth factor (PGF), angiopoietin 1 (ANGPT1), angiopoietin 2 (ANGPT2), erythropoietin (EPO), vascular endothelial growth factor receptor 1 (VEGFR1), fms-related receptor tyrosine kinase 1 (FLT1), and endothelin (EDN1).

A therapeutic composition comprising at least one of the synthetic HIF-1α mRNA of any of the preceding embodiments.

The therapeutic composition of the preceding embodiment, further comprising at least one of a pharmaceutically acceptable carrier.

The therapeutic composition of any preceding embodiments, wherein the at least one of the synthetic HIF-1α mRNA is complexed or associated with at least one of a cationic lipid or an ionizable lipid.

The therapeutic composition of any preceding embodiments, wherein the at least one of the cationic lipid or the ionizable lipid form a lipid-based carrier selected from the group consisting of a liposome and a lipid nanoparticle (LNP).

The therapeutic composition of the preceding embodiment, wherein the lipid-based carrier additionally comprises at least one of a neutral lipid, a steroid or steroid analogue, or any combination thereof.

The therapeutic composition of any preceding embodiments, wherein the lipid-based carrier is a liposome.

The therapeutic composition of the preceding embodiment, wherein the liposome is lipofectamine.

The therapeutic composition of any preceding embodiments, wherein the LNP comprises a PEGylated lipid.

The therapeutic composition of any preceding embodiments, wherein the at least one of the synthetic HIF-1α mRNA is complexed with at least one histidine-lysine co-polymer (HKP) to form a polymeric nanoparticle.

The therapeutic composition of the preceding embodiment, wherein the polymeric nanoparticle further comprises at least one ionizable lipid.

A Kit comprising the synthetized HIF-1. mRNA of any preceding embodiments, or the therapeutic composition of any preceding embodiments.

The kit of the preceding embodiment, further comprising technical instructions providing information on administration and dosage of the synthetized HIF-1α mRNA or therapeutic composition.

The kit of any preceding embodiments, wherein the synthetized HIF-1α mRNA or therapeutic composition is lyophilized, and wherein the kit further comprises a solubilizing liquid.

A method for producing the synthetic HIF-1α mRNA includes providing a complementary deoxyribonucleic acid (cDNA) encoding at least one of a polypeptide derived from a HIF-1α protein or a fragment thereof, and contacting the provided cDNA with an RNA polymerase in the presence of nucleotides, under conditions such that the mRNA is synthesized.

The method of the preceding embodiment, wherein the RNA polymerase is a T7 RNA polymerase.

The method of any preceding embodiments, wherein the nucleobase of at least one of the nucleotide is modified by methylation, isomerization or both.

A method for treating a surgical wound or preventing ischemia or necrosis in wound tissue, includes administering to a subject in need an effective amount of the synthetic HIF-1α mRNA of any preceding embodiments, or the therapeutic composition of any preceding embodiments.

The method of any preceding embodiments, wherein the wound is reconstructed with a pedicle flap.

The method of any preceding embodiments, wherein the administering of the synthetic HIF-1α mRNA or the therapeutic composition causes a HIF-1-induced response.

The method of any preceding embodiments, wherein the HIF-1-induced response promotes the neovascularization and/or maintains the vascularization of the distal portion of the flap.

The method of any preceding embodiments, wherein the wound is from a reconstruction after traumatic injury, complex abdominal hernia repair, breast reconstruction after resection for cancer, and plastic surgical procedures including abdominoplasties and facelifts.

The method of any preceding embodiments, wherein the synthetic HIF-1α mRNA or the therapeutic composition is administered subcutaneously, intramuscularly, or transdermally.

The method of any preceding embodiments, wherein the subject is a human being or an animal.

The method of any preceding embodiments, wherein the human being has a medical condition that increases the risk of ischemia or necrosis during wound healing.

The method of any preceding embodiments, wherein the animal is a cat or a dog and has undergone surgical sterilization.

The synthetic HIF-1α mRNA of any preceding embodiments or the therapeutic composition of any preceding embodiments for use in the treatment of a surgical wound in a subject in need.

The synthetic HIF-1α mRNA or the therapeutic composition for use according to the preceding embodiment, wherein the surgical wound is reconstructed with a pedicle flap.

The synthetic HIF-1α mRNA or the therapeutic composition for use according to any preceding embodiment, wherein the pedicle flap is at risk of ischemia or necrosis.

### DESCRIPTION OF THE FIGURES

FIG. 1A-1C show the locations of HIF-1α injections on the dorsum of rats and the increase of HIF-1α mRNA expression assessed by qRT-PCR after HIF-1α DNA transfection compared to sham control overtime

FIG. 2A-2C provide digital photographic assessments of skin flaps treated with HIF-1a DNA plasmid overtime

FIG. 3A-3B show the in vitro production of 4 HIF-1α mRNA transcript isoforms; full-length predominant form has an open reading frame of 826 amino acids, while variants 1, 2, and 3 contained 424, 516, and 785 amino acids

FIG. 4A-4B show graphs of the relative HIF-1α transcript values of full-length HIF-1a and variant 1 (V1 comprising 424 aa) HIF-1α after transfection into primary dermal fibroblasts (FIG. 4A) and the quantitative expression levels of VEGF, PDG, and ANG-1 (FIG. 4B)

FIG. 5A-5D show that predominant and variant 2 had significantly increased levels of HIF-1α (FIG. 5A) compared to control across all three time points and significant increases of VEGF (FIG. 5B), PGF (FIG. 5C), and ANGPT1 (FIG. 5D) compared to control across all time points for variants 1 and 2.

FIG. 6 shows synthetic HIF-1α mRNA levels of different embodiments after transfection into primary dermal fibroblasts compared to the Lipofectamine only control

FIG. 7 shows the transcriptional stimulation of synthetic HIF-1α mRNA of different embodiments on VEGF in primary dermal fibroblasts compared to the Lipofectamine only control

FIG. 8 shows the transcriptional stimulation of synthetic HIF-1α mRNA of different embodiments on PGF in primary dermal fibroblasts compared to the Lipofectamine only control.

FIG. 9 shows the transcriptional stimulation of synthetic HIF-1α mRNA of different embodiments on ANGPT1 in primary dermal fibroblasts compared to the Lipofectamine only control.

FIG. 10 shows the transcriptional stimulation of synthetic HIF-1α mRNA of different embodiments on FLT1 in primary dermal fibroblasts, compared to the Lipofectamine only control.

FIG. 11 shows graphs of the EDN1 mRNA levels overtime after HIF-1α mRNA transfection into primary dermal fibroblasts, compared to the Lipofectamine only control.

FIG. 12 shows the various structural motifs and functional areas of the full-length predominant HIF-1α protein (top line) and the altered HIF-1α proteins.

### DETAILED DESCRIPTION

All publications and patent applications identified herein are incorporated by reference in their entirety.

Some embodiments of the current subject matter are discussed in detail below. In describing embodiments, specific terminology is employed for the sake of clarity. However, the embodiments are not intended to be limited to the specific terminology so selected. A person skilled in the relevant art will recognize that other equivalent components can be employed, and other methods developed, without departing from the broad concepts of the present subject matter. All references cited anywhere in this specification are incorporated by reference as if each had been individually incorporated.

Unless defined otherwise, technical and scientific terms used herein have the same meaning as commonly understood by a person of ordinary skill in the art. The following definitions are provided to facilitate understanding of certain terms used frequently herein and are not meant to limit the scope of the present disclosure.

Certain nucleotide (RNA or DNA) and amino acid sequences (polynucleotides and polypeptides) as part of the embodiments. It is to be understood that the specifically identified sequences describe other sequences that contain less than 100% sequence identity while retaining the same transcriptional activity or functionality. For example, a nucleotide (RNA or DNA) sequence may have at least 90% sequence identity or at least 95% sequence identity with a polynucleotide specifically disclosed herein and still encodes for an entirely equivalent or functionally equivalent polypeptide. Similarly, a polypeptide may contain less than 100% sequence identity to a polypeptide specifically identified herein and still retains the same transcriptional activity or functionality. For example, a polypeptide may have at least 90% sequence identity or at least 95% sequence identity with a polypeptide specifically disclosed herein and still retains the same or a substantially similar transcriptional activity or functionality as the specifically identified polypeptide.

As used throughout, the term “fragment” refers to a polypeptide derived from the full-length HIF-1α protein or one of its isoforms and is defined as any discrete portion of HIF-1α that retains at least a fraction of the transcriptional activity of the full-length HIF-1α. Preferably the fraction of transcriptional activity retained is at least 90%, at least 95% or 100% of the activity of the full-length protein. It may include amino acid sequences that have conservative variations. The terms fragment and polypeptide may be used interchangeably. In the context of the embodiments, a HIF-1α fragment is a polypeptide capable of eliciting the transcriptional activation of HIF-1-inducible target genes, including the transcription of genes encoding angiogenic factors such as vascular endothelial growth factor (VEGF) and/or proteins involved in wound healing processes including cell migration, cell invasion and/or epithelial-mesenchymal transitions or even local inflammation.

As used throughout, the term “wound” refers to an injury which compromises the integrity of skin tissue or other tissue of the body. The term “surgical wound” refers to an incision through the skin and soft tissue that has been performed to facilitate a surgical procedure.

As used throughout, the term “pedicle flap” refers to a tissue being transfer from one part of the body to another by rotating around an intact vascular pedicle. The flap remains attached to its blood supply, with the tissue surgically tunneled underneath the skin to the area receiving the flap. The rotation of the flap is preferably performed such as to prevent interference with the bloodstream. In certain embodiments, the pedicle flap carries tissue layers such as skin, subcutaneous tissue, fat tissue and optionally some skeletal muscle to preserve the vascular supply. Many surgical procedures rely on pedicle flaps, including reconstruction after traumatic injury in both the military and civilian sectors, complex abdominal hernia repair, breast reconstruction after resections for cancer, oral and maxillofacial reconstruction, and plastic surgical procedures including abdominoplasties and facelifts. For example, in post-mastectomy breast reconstruction, autologous flap not only prevents the risk of complications associated with implants but also provides a long-lasting, natural aesthetic and the best opportunity for sensory restoration favored by patients. However, risks for developing distal flap ischemia or necrosis and even infections account for most complications in patients undergoing pedicle flap-based reconstruction. Such complications can be exacerbated in patients having other medical issues, including diabetes, obesity, aging or a weakened immune system such as in autoimmune disease or cancer. Inadequate or compromised vascularization of pedicle flaps may result in ischemia, and prolonged ischemia can result in partial or complete flap necrosis, leading to flap failure.

As used throughout, the term “ischemia” refers to a restriction in blood supply to portions of the pedicle flap, thereby causing a shortage of oxygen which is needed for cellular metabolism. In certain embodiments, ischemia in a pedicle flap is caused by an inadequate maintenance of the microvascular network and compromised angiogenic processes of the flap, in particular in the distal portion of the flap. Ischemic wounds show reduced adaptative responses to hypoxia and impaired healing, and if left unaddressed may lead to partial or complete necrosis of the pedicle flap.

As used throughout, the term “necrosis” refers to cell injury which results in the premature death of cells in flap tissue, due to a lack of adequate oxygenation and blood supply. Necrosis is a common complication of pedicle flaps and is the result of prolonged and severe ischemia. In certain embodiment, necrosis affects the pedicle partially, especially in its distal flap portion. In other embodiments, the necrosis affects the pedicle flap in its entirety, leading to the failure of the flap.

As used throughout, the term “hypoxia” refers to a state in which oxygen is not available in sufficient amounts in the distal portion or the totality of a flap to maintain adequate homeostasis. In certain embodiments, the inadequate oxygen delivery to the flap is caused by compromised microvasculature which affects the blood supply. In other embodiments, the inadequate oxygen delivery to the flap is due to low oxygen level in the blood. Hypoxia promotes vascularization remodeling by regulating angiogenic pathways which mediate key aspects of endothelial, stromal, and vascular biology. In addition, hypoxia influences additional aspects of angiogenesis, including neovascularization with the sprouting of new blood vessels from existing vessels, tube formation, maturation and function of blood vessels. In contrast, the term “normoxia” refers to a state in which oxygen is available in sufficient amount in the flap, thus resulting in proper wound healing.

Transcriptional responses to hypoxia are mediated by the transcription factor hypoxia-inducible factor 1 (HIF-1) that orchestrates the expression of a plethora of HIF-1-inducible genes, many of which are critical for angiogenesis, inflammation, metabolism, and homeostasis. Hypoxia-inducible factor-1α (HIF-1α) is a subunit of the heterodimeric HIF-1. Under hypoxia, HIF-1α is stabilized and bound to the HIF-1β subunit, which is constitutively expressed to form the active transcription factor HIF-1. After translocation to the nucleus, HIF-1 binds to hypoxia regulatory elements (HREs) within the promoter region of HIF-1-inducible genes. In ischemic or necrotic pedicle flaps, local hypoxia may not be sufficient to induce the degree of angiogenesis needed for maintaining vascularization and/or promoting neovascularization.

The discovery of HIF-1α has presented a new direction for ischemic preconditioning of surgical flaps. As a master regulator promoting the expression of hundreds of growth factors, cytokines, and enzymes, HIF-1α allows the tissue to withstand ischemia. In normoxia, HIF-1α levels are low because it is easily oxidized and degraded. Under hypoxic conditions, however, oxidative destruction of HIF-1α is decreased, and HIF-1α expression is increased. Elevating levels of HIF-1α medically induce angiogenesis. Takaku et al. have explored novel approaches to surgically preconditioning tissue and promote viability of flap in plastic surgical procedures. They have shown that elevating levels of HIF-1α promotes tissue survival in a mouse model. Their approach to elevating HIF-1α levels used systemic treatment with dimethyloxalylglycine (DMOG).

As used throughout, the term “angiogenic factors” refers to growth factors which regulate angiogenesis, the process responsible for neovascularization and remodeling of vasculature in tissue. Angiogenesis is affected by oxygen levels. In pedicle flaps, hypoxia stimulates the development and remodeling of the microvasculature through the activation of HIF-1-inducible angiogenic genes, thus enabling adequate oxygen delivery to oxygen-deprived portion such as the distal portion of flaps. Examples of HIF-1-inducible angiogenic factors include, but are not limited to vascular endothelial growth factor (VEGF), angiopoietin-1, angiopoietin-2, platelet-derived growth factor (PDGF), basic fibroblast growth factor (bFGF), and monocyte chemoattractant protein-1 (MCP-1). Angiogenic growth factors activate endogenous endothelial cells and also recruit a heterogenous population of cells that participate in various aspects of angiogenesis. For instance, VEGF acts in a paracrine manner to stimulate the differentiation, proliferation and survival of endothelial cells, leading to sprouting of new blood vessels

In addition, HIF-1 regulates the expression of genes involves in wound repair, including Vascular Endothelial Growth Factor, Placental Growth Factor, Angiopoietin 1, Vascular endothelial growth factor receptor 1, and Endothelin 1 and/or genes involve in cell migration, proliferation, and differentiation.

As the main regulator of oxygen homeostasis, HIF-1α is a therapeutic target for regulating the expression of genes involved in angiogenesis and wound healing. Utilizing HIF-1a for its angiogenic properties improves flap survival and viability.

Synthetic HIF-1α mRNAs

In certain embodiments, a synthetic HIF-1α mRNA comprises an open reading frame encoding at least one of a polypeptide derived from a HIF-1α protein or a fragment thereof, wherein the polypeptide or fragment thereof elicits the expression of at least one of an angiogenic factor involved in wound healing. The synthetic HIF-1α mRNA of the preceding embodiments may comprise an open reading frame comprising at least one of a nucleic acid sequence having at least 90%, at least 95% or 100% identity with the nucleic acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and ID NO: 24, and a fragment thereof.

In certain embodiments, the synthetic HIF-1α mRNA comprises an open reading frame encoding at least one of a polypeptide derived from HIF-1α protein or one of its isoforms, wherein the polypeptide elicits the expression of at least one of an angiogenic factor involved in wound healing. The synthetic HIF-1α mRNA of any of the preceding embodiments may comprise open reading frame comprising at least a nucleic acid sequence having at least 90%, at least 95% or 100% identity with the nucleic acid sequence SEQ ID NO: 16, SEQ ID NO: 22, SEQ ID NO: 23 or SEQ ID NO: 24 or fragment thereof.

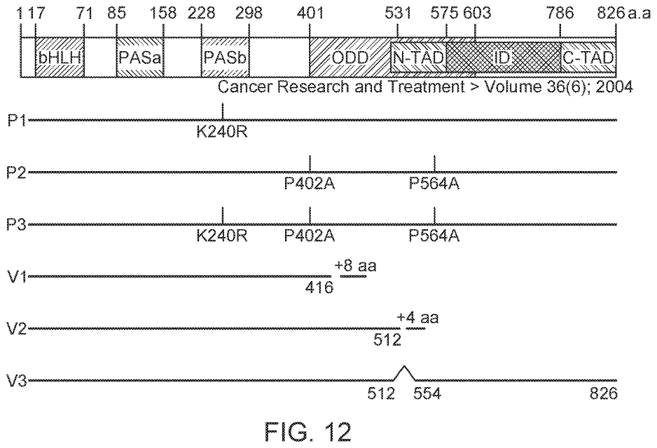

HIF1-α comprises several functional domains including the oxygen-dependent degradation domain (ODDD, amino acids 401-603) which is located in the central portion of the full-length protein and is responsible for its oxygen-dependent degradation. (See FIG. 12) In normoxia, HIF-1α is polyubiquitinated at 2 conserved proline amino acid residues within ODDD by prolyl hydroxylases (PHD). Hydroxylated prolines are recognized by the von Hippel-Lindau (VHL) E3 ubiquitin ligase complex that ubiquitinates HIF-1α, thereby targeting it for proteasomal degradation. Since the hydroxylation reaction requires oxygen as a substrate, hypoxia limits the reaction, thereby attenuating HIF-α hydroxylation. HIF-α stabilizes and accumulates in the cells and subsequently translocates into the nucleus, where it dimerizes with HIF-1β and activates the transcription by binding to HREs in the promoter region of the target genes needed for oxygen homeostasis.

In certain embodiments, the synthetic HIF-1α mRNA comprises an open reading frame comprising at least one of a mutation encoding for an amino acid residue susceptible to hydroxylation under conditions of normoxia. The at least one of a mutation prevents hydroxylation and as such impairs HIF1-α ubiquitin-dependent degradation and extends the protein half-life without affecting its transcriptional activity. In one aspect of the invention, the amino acid residue is a proline. In another aspect of the invention, the amino acid residue is the proline residue at position 564 and/or the proline residue at position 402 of the ODDD, when the numbering of the amino acid residues is that of the human HIF-1α protein. In yet another aspect of the embodiments, both proline residues are mutated to prevent hydroxylation, thereby stabilizing the polypeptide in the presence of oxygen. Both the proline residues at position 564 and 402 of the ODDD are mutated, when the numbering of the amino acid residues is that of the human HIF-1α protein as defined in SEQ ID NO: 10. (See FIG. 12 and Table 1) In such embodiments, the synthetic HIF-1α mRNA comprises an open reading frame comprising at least one of a nucleic acid sequence having at least 90%, at least 95% or 100% identity with the nucleic acid sequence SEQ ID NO: 17 or SEQ ID NO: 18 while retaining its ability to elicit the expression of at least one of an angiogenic factor involved in wound healing and/or at least one of a protein regulating cell migration, proliferation and differentiation.

TABLE 1
Description of the sequences and their respective mutations and/or
deletions with respect to the sequences of wild type HIF-1α

| | Nucleic acid | mRNA | AA |
|---|---|---|---|
| Name | mutations/deletion | | mutations/deletion |
| | SEQ ID NO | | SEQ ID NO |
| P1 | 1011: A to G | SEQ ID NO: 16 | Lys240Arg |
| 5.1 | 2280: A to G | | Gln663Arg |
| | SEQ ID NO: 1 | | SEQ ID NO: 10 |
| 5-1 3MA | 1011: no mutation | SEQ ID NO: 17 | Pro402Ala |
| P2 | 1496: C to G | | Pro564Ala |
| | 1982: C to G | | Gln663Arg |
| | 2280: A to G | | |
| | SEQ ID NO: 2 | | SEQ ID NO: 11 |
| 5-1 2MA | 1011: A to G | SEQ ID NO: 18 | Lys240Arg |
| P3 | 1496: C to G | | Pro402Ala |
| | 1982: C to G | | Pro564Ala |
| | 2280: A to G | | Gln663Arg |
| | SEQ ID NO: 3 | | SEQ ID NO: 12 |
| 5-4 | 873: G to A | SEQ ID NO: 19 | Cys194Tyr |
| V1 | 941: A to G | | Met217Val |
| | Deletion: 1541-2384 | | Ser to Ile |
| | 2409: stop TAA | | |
| | 2593: G to A | | |
| | SEQ ID NO: 4 | | SEQ ID NO: 13 |
| 8-2 | 1365: 1 to G | SEQ ID NO: 20 | Glu358Gly |
| V2 | Deletion: 1829-2385 | | |
| | 2398: Stop TGA | | |
| | SEQ ID NO: 5 | | SEQ ID NO: 14 |
| 8-3 | 1513: A to G | | Thr |
| V3 | Deletion: 1829-1951 | | Deletion: 41 aa |
| | 2433: A to T | | Gln714Leu |
| | 2558: A to G | | Lys756Glu |
| | SEQ ID NO: 6 | SEQ ID NO: 21 | SEQ ID NO: 15 |
| NM_001530.4 | | transcript variant 1 | Isoform 1 |
| | SEQ ID NO. 7 | SEQ ID NO. 22 | |
| NM_181054.3 | lacks coding exon | Transcript variant 2 | Isoform 1.2 |
| | SEQ ID NO: 8 | SEQ ID NO. 23 | |
| NM_001243084.2 | alternate 5′term. exon | Transcript variant 3 | Isoform 1.3 |
| | SEQ ID NO: 9 | SEQ ID NO. 24 | |

In certain embodiments, the synthetic HIF-loa mRNA comprises an open reading frame comprising a deletion which encompasses at least part of the oxygen-dependent degradation domain (ODDD) to impair the ubiquitin-dependent degradation of the HIF-1α polypeptide while retaining its transcriptional activity. (See FIG. 12) Such deletion results in the stabilization of HIF-1α and enables the transcription of HIF-1α-inducible genes under normoxia, amid the presence of oxygen. In such embodiment, the synthetic HIF-1α mRNA comprises an open reading frame comprising at least one of a nucleic acid sequence that has at least 90%, at least 95% or 100% identity with the nucleic acid sequence SEQ ID NO: 19, SEQ ID NO: 20 or SEQ ID NO: 21. HIF-1α mRNA (5.4) comprising SEQ ID NO: 19, HIF-1α mRNA (8.2) comprising SEQ ID NO: 20 and HIF-1α mRNA (8.3) comprising SEQ ID NO: 21, all have a deleted portion of ODDD. Interestingly, all 3 HIF-1α mRNA carry an unmutated proline at position 402 while (5.4) and (8.2) lack the proline at position 564. (8.3) has an unmutated proline at position 564. In some embodiments, the deletions encompass at least one of a domain of HIF-1α which affect the function, regulation, heterodimerization of HIF-1α or HIF1-α interaction with at least one of its partners. In certain aspects of the embodiments, the deletions do not affect the ability of HIF-1α mutant to elicit the expression of at least one of an angiogenic factor involved in wound healing.

Synthesis of HIF-1α mRNAs

The HIF-1α mRNA of any of the preceding embodiments may be synthesized using any known methods in the art. In certain embodiments, the HIF-1α mRNA is synthesized by in vitro transcription (IVT). Briefly, IVT is performed by providing a complementary deoxyribonucleic acid (cDNA) encoding at least one of a polypeptide derived from a HIF-1α protein, and contacting the provided cDNA with an RNA polymerase in the presence of nucleotides, under conditions such that the mRNA is synthesized. For example, RNA polymerase includes, but not limiting to T3, T7 or SP6 RNA polymerase.

The HIF-1α mRNA of any of the preceding embodiments may be synthesized as an unmodified or a modified mRNA. Typically, mRNAs are modified to enhance stability, reduce innate immune response and improve translation. Modifications of mRNAs include, but are not limited to modifications of the nucleotides such as backbone modifications, sugar modifications or nucleobase modifications. In certain embodiments, the nucleobase of at least one of the nucleotides of the synthetic HIF-1α mRNA is modified by methylation, isomerization or both. For example, the modified nucleobase may be selected from the group consisting of pseudouridine (PsU), N1-methyl-pseudouridine (1mPsU), 5-methyl-uridine (5mU), 2′-O-methyl-uridine (2′-O-methyl-U), 5-methyl-cytidine (5mC), 2-thiouridine (s2U), N6-methyladenosine (6 mA), and any combination thereof. Preferably, the modified nucleobase is N1-methyl-pseudouridine (1mPsU). The synthetic HIF-1α mRNA of any of the preceding embodiments may include RNA backbone modifications in which the phosphates are modified chemically. In certain embodiments, the HIF-1α mRNA may contain sugar modifications which typically involves 2′-O-modification of the ribose. Such modifications have been shown to enhance the thermodynamic stability of the synthetic mRNA.

The synthetic HIF-1α mRNAs of any of the preceding embodiments may further includes at least one of a poly-A tail, a methylated cap, a 5′ untranslated region (5′-UTR), a 3′ untranslated region (3′-UTR) or any combination thereof. The presence of a poly-A tail and/or a cap is not essential for the functionality of the synthetic mRNA but may improve the efficiency of the translation and provide protection against from degradation. In one aspect of the embodiments, the synthetic HIF-1α mRNA includes a 5′ cap structure. Examples of cap structures include, but are not limited to, m7G(5′)ppp (5′(A,G(5′)ppp(5′)A and G(5′)ppp(5′)G. In another aspect of the embodiments, the synthetic HIF-1α mRNA includes a tail structure which typically protects the mRNA from exonuclease degradation. The poly-A tail is thought to stabilize synthetic mRNAs. Therefore, a long poly A tail of at least about 20, 50, 100, 200, 300, 400 at least 500 nucleotides can be added on the 3′ terminus using a variety of art-recognized techniques.

The synthetic HIF-1α mRNA of any of the preceding embodiments may further include at least one of a 5′ and/or 3′ untranslated region. Typically, a 5′ untranslated region includes one or more elements that affect mRNA's stability or translation efficiency, for example, an iron responsive element. Typically, a 3′ untranslated region includes one or more of a polyadenylation signal or a binding site for proteins that affects an mRNA's stability.

Transcriptional Activity of Synthetic HIF-α mRNAs

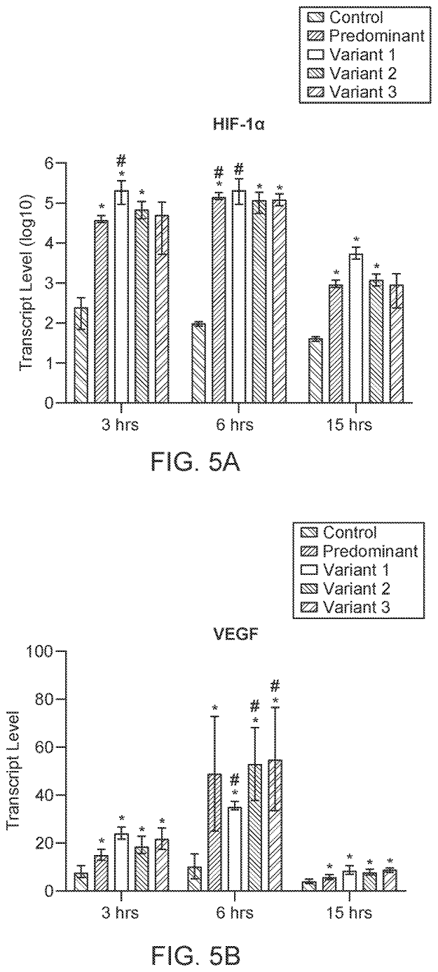

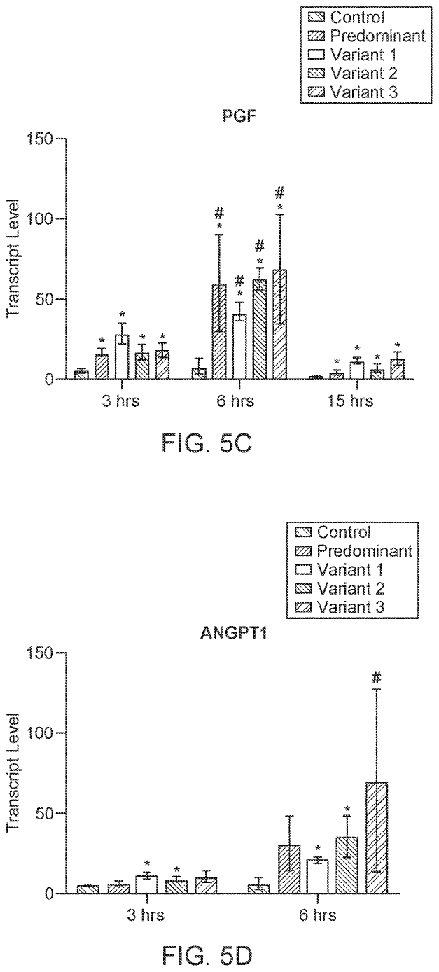

The synthetic HIF-1α mRNA of any of the preceding embodiments may elicit the expression of at least one of an angiogenic factor involved in wound healing. (FIG. 5-11) In certain embodiments, the wound is caused by a surgical procedure and is reconstructed with a pedicle flap.

In certain embodiments, the at least one of an angiogenic factor is selected from the group of consisting of vascular endothelial growth factor (VEGF), placental growth factor (PGF), angiopoietin 1 (ANGPT1), angiopoietin 2 (ANGPT2), erythropoietin (EPO), vascular endothelial growth factor receptor 1 (VEGFR1), fins-related receptor tyrosine kinase 1 (FLT1), and endothelin (EDN1). (FIG. 5-11)

In certain embodiments, pedicle flaps, in particular the distal portion of the flaps are at risk for ischemia or even necrosis when the vasculature is compromised. The vasculature becomes inadequate and/or compromised to provide oxygen and nutrient to the distal portions. In one aspect of the embodiments, the synthetic HIF-1α mRNA upregulates the expression of angiogenic factors which facilitates the maintenance and/or remodeling of the vasculature in the flap. Accordingly, the synthetic HIF-1α mRNA enhances flap survival and viability. Maintaining vascularization and/or promoting neovascularization using synthetic HIF-1α mRNA of any of the preceding embodiments in pedicle flaps facilitates wound healing.

The synthetic HIF-1α mRNA of any of the preceding embodiments may elicit the expression of at least one of the genes modulating cell migration, cell invasion and/or epithelial-mesenchymal transitions including but limited to matrix metalloproteinases, E-Cadherin and/or at least genes regulating vascular mimicry including but not limiting to VE-Cadherin, EphA2, LN5 gamma2.

Please note that this invention is not limited to the expression of angiogenic factors. Other proteins/polypeptides regulating various wound healing processes may have their expression upregulated if their gene expression is modulated by HIF-1α, including but not limited to proteins involve in cell migration, proliferation, differentiation or inflammation.

### Therapeutic Compositions

At least one of the synthetic HIF-1α mRNA of any of the preceding embodiments may be formulated in a therapeutic composition. In one aspect of the embodiments, the therapeutic composition further includes at least one of a pharmaceutically acceptable carrier. In certain embodiments, the at least one of the synthetic HIF-1α mRNA is complexed or associated with at least one of a cationic lipid or an ionizable lipid. The at least one of the cationic lipid or the ionizable lipid forms a lipid-based carrier. In one aspect of the embodiments, the lipid-based carrier is a liposome i.e., bilayer membrane which transports the mRNA within its aqueous core, complexed within the bilayer membrane, or associated with the exterior surface of the liposome. Typically, the size of the liposomes varies between 50 and 1,000 nm. In another aspect of the embodiments, the lipid-based carrier is lipid nanoparticle (LNP) which typically displays a micelle-like structure, encapsulating the synthetic HIF-1α mRNA in a non-aqueous core. The size of LNPs varies between 20 and 150 nm The purpose of encapsulating the synthetic HIF-1α mRNA is to protect the nucleic acid from degradation. The processes of loading an mRNA onto a liposome or encapsulating a mRNA into a LNP are well-known in the art. (See Lipid nanoparticles for mRNA delivery. Xucheng Hou, Tal Zaks, Robert Langer & Yizhou Dong. Nature Reviews Materials volume 6, pages 1078-1094 (2021). Accordingly, in the context of the embodiments, the lipid-based carrier is capable of enhancing the stability of the synthetic HIF-1α mRNA in vivo and/or facilitate its delivery to the target cells of the pedicle flap.

Cationic lipids are essential for lipid-base carriers but several are associated with cytotoxicity. In certain embodiments, the at least one of the cationic lipids is an ionizable lipid which circumvents cytotoxicity as it has near-neutral charge at physiological pH, forming reverse micelles that encapsulate the synthetic HIF-1α mRNA in an LNP. Several cationic lipids have been described in the literature, many of which are commercially available. Particularly suitable cationic lipids for use in the therapeutic composition of any of the preceding embodiments may be N-[1-(2,3-dioleyloxy)propyl]-N,N,N-trimethylammonium chloride (DOTMA). DOTMA may be formulated alone or may be combined with the neutral lipid, dioleoylphosphatidyl-ethanolamine (DOPE) or other cationic or non-cationic lipids into a liposome or an LNP.

Suitable ionizable lipids include, but are not limited to, 5-carboxyspermylglycinedioctadecylamide or (DOGS), 2,3-dioleyloxy-N-[2(spermine-carboxamido)ethyl]-N,N-dimethyl-1-propanaminium or DOSPA, 1,2-Dioleoyl-3-Dimethylammonium-Propane (DODAP), 1,2-Dioleoyl-3-Trimethylammonium-Propane (DOTAP). Preferably, when the lipid-based carrier is a liposome, the liposome is lipofectamine which consists of a 3:1 mixture of DOSPA (2,3-dioleoyloxy-N-[2(sperminecarboxamido)ethyl]-N,N-dimethyl-1-propaniminium trifluoroacetate) and DOPE.

Additional ionizable lipids also include, but are not limited to 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylarnrnonium bromide (DDAB), N41,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy 1-1-(cis,cis-9′, 1-2′-octadecadienoxy)propane (CpLinDMA), N,N-dimethyl-3,4-dioleyloxybenzylamine or DMOBA, 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinolcylcarbamyl-3-dimethylaminopropane (DLincarbDAP), 1,2-Dilinolcoylcarbamyl-3-dimethylaminopropane (DLinCDAP), 2,2-dilinoleyl-4-dimethylaminomethyl-[1,3]-dioxolane (DLinDMA), 2,2-dilinoleyl-4-dimethylaminoethyl-[1,3]-dioxolane (DLin-K-XTC2-DMA), and 2-(2,2-di((9Z,12Z)-octadeca-9,1 2-dien-1-yl)-1,3-dioxolan-4-yl)-N,N-dimethylethanamine or (DLin-KC2-DMA).

In certain embodiments, the lipid-based carrier additionally comprises at least one of a neutral lipid, a steroid or steroid analogue, a PEG-lipid or any combination thereof.

In certain embodiments, the at least one of a neutral lipid may be any zwitterion. Several neutral lipids have been described in the literature, many of which are commercially available. Particularly suitable neutral lipids for use in the therapeutic composition of any of the preceding embodiments may include, but are not limited to distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In certain embodiments, the at least one of a steroid or steroid derivative may provide structural integrity and functions to increase the stability of the lipid-based carrier, reduce the mobility of phospholipids, reduce leakage of the encapsulated synthetic HIF-1α mRNA, and increase the efficiency of endosome escape. Examples of steroid may include, but are not limited to cholesterol, coprosterol, sitosterol, ergosterol, campesterol, stigmasterol, brassicasterol, tomatidine, lycopersicin, ursolic acid, alpha-tocopherol, and corticosteroid.

In certain embodiments, the lipid-based carrier additionally comprises at least one of a polyethylene glycol (PEG)-modified lipid which may prevent aggregation and increase circulation half-life in vivo. Examples of PEG-lipid may include, but are not limited to PEG-modified phosphatidylethanolamine, PEG-modified phosphatidic acid, PEG-modified ceramide, PEG-modified dialkylamine, PEG-modified diacylglycerol, and PEG-modified dialkylglycerol. In one aspect of the embodiments, PEGylated lipids may also comprise functional groups, such as amine or maleimide, which are used to conjugate molecules to improve cellular targeting.

In certain embodiments, the at least one of the synthetic HIF-1α mRNA is formulated in nanoparticles which do not contain PEG-lipid.

In certain embodiments, the at least one of the synthetic HIF-1α mRNA is complexed with at least one histidine-lysine co-polymer (HKP) to form a polymeric nanoparticle. In one aspect of the embodiments, the HKP comprises H3K(+H)4b, H3k(+H)4b or both. The HKP-based nanoparticle functions as a delivery system and allows an efficient systemic delivery of the synthetic HIF-1α mRNA of any of the embodiments. In certain embodiments, a stable PLNP consists of a positively charged HKP complexed with a negatively charged HIF-1α mRNA through electrostatic interaction and hydrogen bonds. In one aspect of the embodiments, the HKP is cross-linked through covalent bond. In another aspect of the embodiments, the HKP has a positive charge and a functional group which can be further modified for targeting specificity and reducing toxicity.

In certain embodiments, the at least one of the synthetic HIF-1α mRNA is formulated in polypeptide-lipid nanoparticles (PLNP). In one aspect of the embodiments, the PLNP is a HKP-based nanoparticle further comprising at least one of a lipid, optionally a cationic lipid and/or an ionizable lipid to form a polypeptide-lipid nanoparticles (PLNP). In one aspect of the embodiments, the PLNP further comprises at least one of a neutral lipid, a steroid or steroid analogue, or any combination thereof, optionally a PEGylated lipid. Such PLNP comprising at least one of the synthetic HIF-1α mRNA of any of the embodiments has lower toxicity and higher efficient cellular delivery through better endosomal escape. In some aspects of the embodiments, the PLNP does not include polyethylene glycol (PEG), which may potentially cause adverse reactions in some patients PLNP may advantageously be manufactured more easily than LNP delivery platforms due to fewer components. Notably, the synthetic HIF-1α mRNA of the embodiments, formulated as PLNP are stable at ambient temperatures, thus eliminating distribution costs associated with cold chain storage of LNP-based products.

In certain embodiments, the selection of the different components of a liposome and/or an LNP and/or PLNP as well as their relative molar ratio may be optimized according to the structure/PK characteristics of the different lipids, the intended target cells and the characteristics of the synthetic HIF-1α mRNA. By varying lipid ratios and structures, optimized formulation may increase the potency of the lipid-based carriers, such as increasing ionizable lipid:mRNA weight ratios.

### Therapeutic Uses

The invention provides methods for treating a surgical wound or preventing ischemia or necrosis in wound tissue by administering to a subject in need an effective amount of the synthetic mRNA of any of preceding embodiments, or the therapeutic composition of any of the preceding embodiments. The surgical wound may result from reconstruction after traumatic injury, complex abdominal hernia repair, breast reconstruction after resection for cancer, and plastic surgical procedures including abdominoplasties and facelifts. Such surgical wounds are preferably reconstructed with pedicle flaps and may be at risk of ischemia or necrosis. HIF-1α mRNA-based therapy facilitates faster and more complete wound healing but promoting survival of pedicle flaps.

The administration of the synthetic HIF-1α mRNA or the therapeutic composition may partially or completely reduce the severity of, and/or incidence of ischemia or necrosis in pedicle flaps. In certain embodiments, the HIF-1α, mRNA-based therapy may be administered to a subject with ischemic and/or necrotic tissue in the pedicle flap of surgical wound. In other embodiments, the HIF-1α mRNA-based therapy may be administered to a subject known to have increased risk of developing ischemia or necrosis. Such subject may suffer from autoimmune diseases, diabetes, obesity or aging and are at higher risk of complicated and/or lengthen wound healing.

Transfection of HIF-1α mRNA into primary dermal fibroblasts results in a rapid, intense, yet transient elevation of HIF-1-inducible target genes. These genes are vital for the establishment and maintenance of a vascular blood supply. In certain embodiments, the administering of the synthetic mRNA or the therapeutic composition causes an HIF-1-induced response. In one aspect of the embodiments, the administration of the synthetic HIF-1α mRNA as a naked RNA or therapeutic composition induces the transcription of at least one of a HIF-1-inducible target gene. For example, a HIF-1-inducible target gene encodes an angiogenic factor including but not limiting to VEGF, PDGF, angiopoietin 1 and 2, PGF, and other angiogenic peptides that promotes the neovascularization and/or maintains the vascularization of the distal portion of the flap.

The synthetic HIF-1α mRNA-based therapy may be administered subcutaneously, intramuscularly, or transdermally. In certain embodiments, the synthetic HIF-1α mRNA may be injected into and near the wound-healing site. If additional doses are required, these can be administered when needed. In other embodiments, the synthetic HIF-1α mRNA or therapeutic composition may be applied as an ointment to facilitate wound-healing care. Such treatment may become routine not only for use in patients to facilitate healing of pedicle flaps post reconstruction surgery, but even with relatively minor cuts and bruises.

The synthetic HIF-1α mRNA-based therapy may be administered to a human being or an animal. In certain embodiments, synthetic HIF-1α mRNA-based therapy may be used for animal wound healing. A vast majority of cats and dogs undergo surgical neutering operations and may benefit from faster healing following the administration of the synthetic HIF-1α mRNA. The potential market for veterinary use is significant.

In certain embodiments, the synthetized HIF-1α mRNA or the therapeutic composition can be provided as a kit. In one aspect of the embodiments, the kit further includes technical instructions providing information on administration and dosage of the synthetized HIF-1α mRNA or therapeutic composition. In another aspect of the embodiments, the synthetized HIF-1α mRNA or therapeutic composition is lyophilized, and the kit further includes a solubilizing liquid.

### Examples

HIF-1α DNA Transfection Promotes HIF-1α mRNA Expression Assessed by qRT-PCR

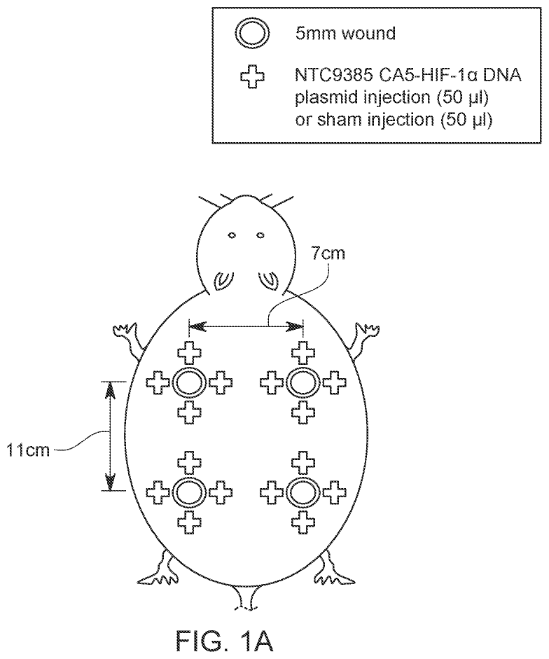

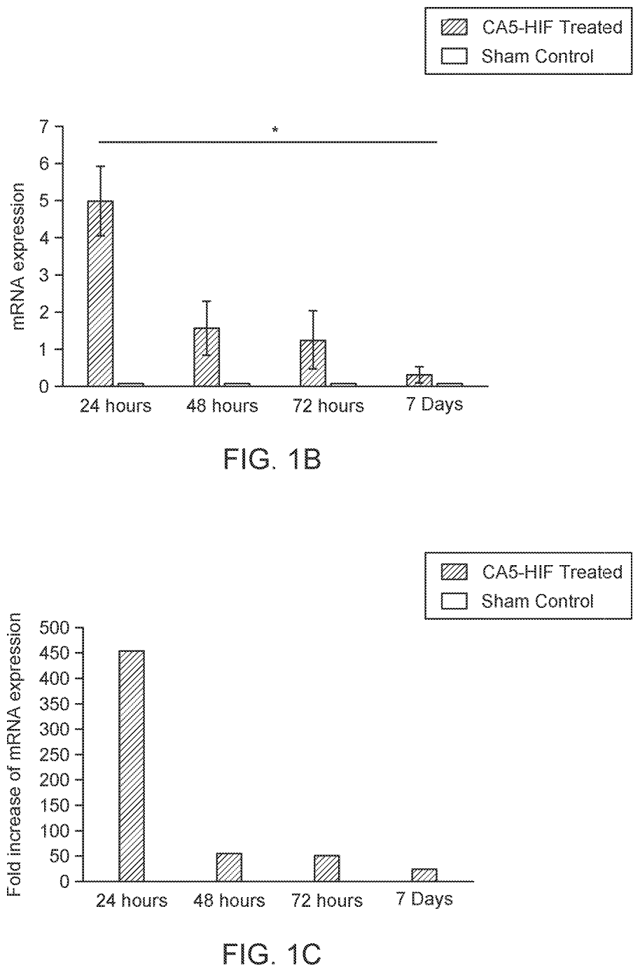

To measure human HIF-1α from the plasmid DNA separately from endogenous rat HIF-1α, specific primers for the detection of human or rat HIF-1α sequences were designed. In initial experiments, HIF-1α plasmid increases levels of human HIF-1α mRNA without increasing the levels of rat HIF-1α mRNA. Then, the production of human HIF-1α mRNA by the DNA plasmid was assessed. Rat dermis was injected with 50 μg of 1 μg/μL plasmid at four marked locations on the dorsum of each of four rats (FIG. 1A). Skin samples were harvested at 24, 48, 72 h, and 7 days. The level of human HIF-1α mRNA expression in HIF-1α injected dermis was significantly higher at 24 and 48 h with 5±0.95-fold and 1.84±0.52-fold increase respectively when compared to sham injected dermis (FIG. 1B). The fold increase of HIF-1α mRNA expression in the HIF-1α transfected cells over sham control cells at 24, 48, 72 h and 7 days was 442, 62, 50, 30 times greater respectively (FIG. 1C). The expression of human HIF-1α mRNA in the rat dermis steadily decreased over the seven-day period.

Treatment of Skin Flaps with HIF-1α DNA Plasmid Improves Tissue Viability According to Digital Photographic Assessment.

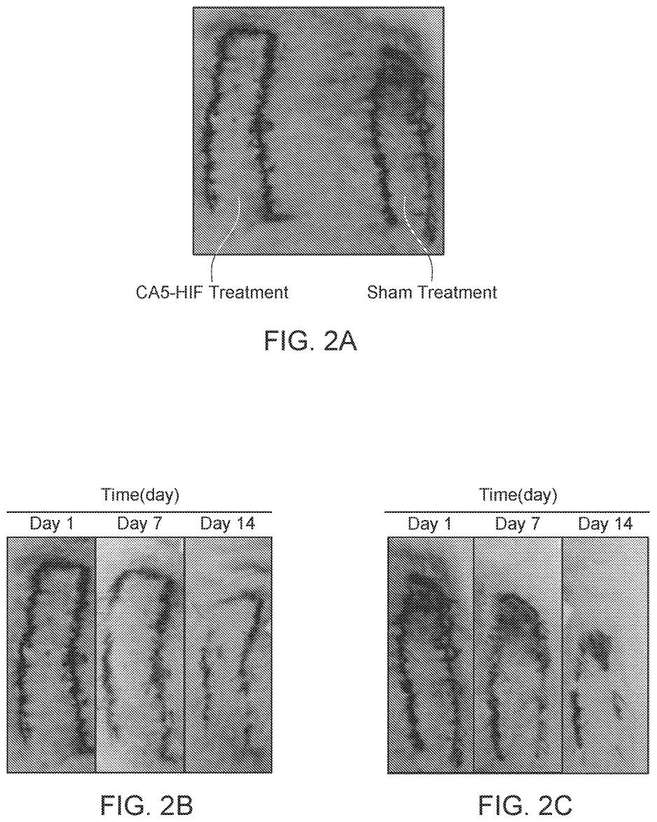

Paired dorsal pedicle flaps were pre-treated with intradermal injections of either saline (sham control) or HIF-1α plasmid, 50 μl of 1 μg/μL. Flap survival was assessed on postoperative days 1, 7, and 14 using digital photography as described above. FIG. 2A shows the progression of skin flaps over a 14-day period of time after the surgery. Treatment with HIF-1α DNA plasmid significantly decreased the area of necrosis on post-surgery days 1 and day 7 (p=0.03 on day 1 and p=0.02 on day 7). On all three days, the area of necrosis of the untreated flap was greater than the treated flap (n=10; p=0.03 on day 1 and p=0.02 on day 7). (FIG. 2B) The treatment of HIF-1α DNA plasmid was significantly effective on days 1, 7, and 14. (FIG. 2C)

### Materials and Methods

Isolation, Cloning, and Production In Vitro of Four HIF-1α mRNA Transcript Isoforms (Predominant and Isoforms 1, 1.2 and 1.3)

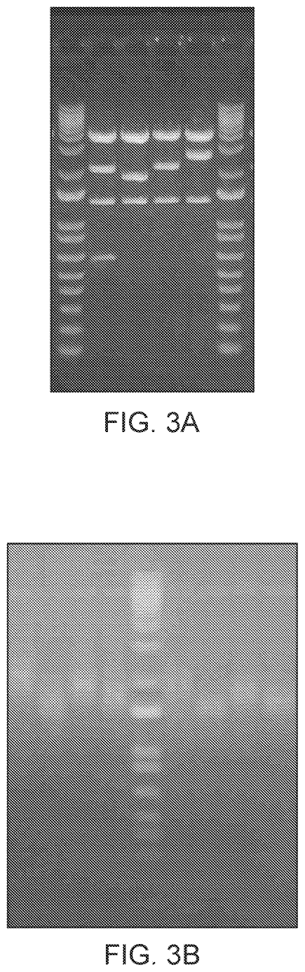

Since HIF-1α is produced at relatively medium-high levels in human white blood cells, a leukocyte cDNA library was purchased from TaKaRa Corporation (Japan). This library contains a high percentage of full-length cDNAs which is important for the cloning of HIF-1α since the full-length transcript is almost 4000 bases long, not including the poly-A tail. DNA PCR primers corresponding to the 5′ and 3′ ends of the HIF-1α transcript were purchased (IDT, Iowa). The 5′ primer contained a 5′-terminal were designed containing a 20 base T7 RNA polymerase promoter. A PCR kit containing Avantage2 Taq polymerase was purchased from TaKaRa and PCR reactions were performed as directed by the manufacturer. The PCR products were cloned using a TA TOPO cloning kit (ThermoFisher, USA). DNA from individual clones was isolated, digested with restriction enzymes and run on agarose gels. Clones with differing restriction enzyme digestion patterns were selected and the plasmid inserts sequenced by Sanger sequencing at the JHU Sequencing Core Facility. A HIF-1α predominant form and three transcriptional variants encoding isoform 1 (SEQ ID NO: 22), isoform 1.2 (SEQ ID NO: 23) and isoform 1.3 (SEQ ID NO: 24)/were documented. (FIG. 3A-3B)

### Mutation of Two Prolines Involved in HIF-1α Protein Degradation

The predominant full-length HIF-1α isoform contains two proline residues known to participate in degradation. V1 and V2 each contains one of these prolines, and V3 contains both of these proline residues. (FIG. 12, Table 1) The GeneArt™ Site-Directed Mutagenesis PLUS System was used to eliminate these prolines from the respective isoform in order to test their abilities to increase HIF-1α response gene levels. The removal of at least of one of the proline residues increases the protein half-life, although it does not render the protein completely resistant to degradation and turn-over.

Oligo primers were designed to change one nucleotide in a particular sequence using PCR amplification. This technique allows up to three different nucleotides being changed in each PCR amplification. The use of in vitro DNA recombination yielded a circular plasmid used to transform competent E. coli cells. Individual clones were selected, the plasmid DNA was extracted and sequenced at the JHU facility to verify that the correct directed mutations had occurred. The Prolines at position 402 and 564 were changed from Proline to Alanine in constucts P2/5.1.3 mA and P3/5.1.2 mA. (FIG. 12, Table 1)

Transfection of Fibroblasts with HIF-1α mRNA Transcripts

Primary human dermal fibroblasts were purchased (ThermoFisher) and grown in cell culture using medium purchased from the manufacturer. Transfection of the fibroblasts with the HIF-1α mRNA transcripts was done with Lipofectamine MessengerMAX Transfection Reagent (ThermoFisher) according to instructions provided by the manufacturer. An extensive series of experiments were completed that determined appropriate amounts of the lipofectamine reagent, as well as varying amounts of HIF-1α mRNA (data not shown). This was used to determine the optimum dose and time required for the maximum increase in the response genes mRNA levels.

### Quantitation of HIF-1α Response Genes Using Reverse Transcriptase Quantitative PCR

At various times post-transfection, the cell culture plate well medium was removed, the well was rinsed with 1×TBS, and buffer RLT from the RNeasy mini kit was directly added to the well. The lysed cell solution was removed, and the total RNA was extracted according to instructions provided by the manufacturer (Qiagen, Maryland). Selected gene specific primers (IDT) were used to determine gene expression levels using real-time quantitative RT PCR using a QuantiNova SYBR Green Probe RT-PCR kit (Qiagen) and the CFX96 Real-Time PCR Detection System (BioRad, CA). All specific gene quantitative values were normalized to actin levels, and the results analyzed using Student's t-test.

First Transfection of HIF-1α mRNA into Primary Fibroblasts

The predominant full-length HIF-1α mRNA and construct (5.4) (424 aa) were transfected into primary dermal fibroblasts (ATCC, Virginia) using an mRNA Lipofectamine kit (ThermoFisher) in triplicate wells of a 96-well plate. Construct (5.4) was selected because it had the largest deletion of the open-reading-frame of the three different constructs e.i., 5.4, 8.2 and 8.3. At 24 hours after transfection, total RNA was extracted (RNeasy mini-kit, Qiagen) and selected gene specific primers (IDT) were used to determine gene expression levels using real-time quantitative RT PCR.

Second Transfection of HIF-1α mRNA into Primary Fibroblasts

Predominant and three variant HIF-1α 5′-methyl-capped poly-A tailed mRNA were synthesized using T7 RNA polymerase. Primary human fibroblasts were transfected using Lipofectamine MessengerMAX in triplicate, and RNA levels were assessed using RT-qPCR at 3-, 6-, and 15-hour transfection time points. All gene transcript levels were normalized to β-actin expression levels.

Third Transfection of HIF-1α mRNA into Primary Fibroblasts

Predominant and three construct HIF-1α 5′-methyl-capped poly-A tailed mRNA were synthesized using T7 RNA polymerase. Primary human fibroblasts were transfected using Lipofectamine MessengerMAX in triplicate. Each well of a 96-well cell culture plate was seeded with 10,000 primary dermal fibroblasts. At 24 hours, each well was transfected using 0.3 ug of the appropriate mRNA and 0.3 ul of the lipofectamine. At the designated times, total RNA was extracted and specific gene mRNA levels were assessed using RT-qPCR at 3-, 6-, and 10-hour transfection time points. All gene transcript levels were normalized to β-actin expression levels and analyzed by Student's t test.

### Results

Isolation, Cloning, and Production In Vitro of Four HIF-1α mRNA Transcript Isoforms

A leukocyte cDNA library was purchased from TaKaRa Corporation (Japan). DNA PCR primers (IDT, Iowa) were designed containing a 20 base T7 RNA polymerase promoter. The PCR products cloned using a TA TOPO cloning (ThermoFisher, USA). DNA from individual clones was isolated, digested with restriction enzymes and run on agarose gels (FIG. 3A, lanes 2, 3, 4, 5). A full-length “predominant’ form as well as three transcript variants were isolated. Sanger DNA sequencing covered 100% of each HIF-1α isoform. The full-length predominant form contained an open reading frame of 826 amino acids, while variants 1/(5.4), 2/(8.2), and 3/(8.3)/contained 424, 516, and 785 amino acids respectively. The HiScribe T7 ARCA mRNA kit (NEB, Mass.) yielded transcripts with a 5′-methyl-cap and a 3′ poly-A tail. Each mRNA reaction was purified (RNA Cleanup, NEB) quantitated, and visualized on an agarose gel (FIG. 3B, lanes 1, 2, 3, 4) are before purification, (lanes 6, 7, 8, 9) are after purification).

First Transfection of HIF-1α mRNA into Primary Dermal Fibroblasts

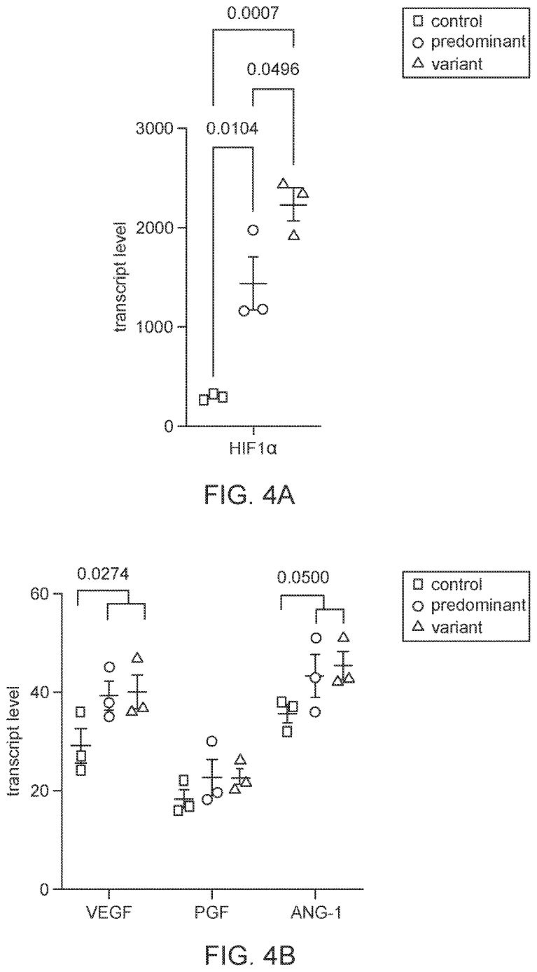

The predominant full-length HIF-1α mRNA transcript and variant #1 (424 aa) were transfected into primary dermal fibroblasts (ATCC, Virginia) using an mRNA Lipofectamine kit (ThermoFisher) in triplicate wells of a 96-well plate. FIG. 4A shows the relative HIF-1α transcript values, and FIG. 4B shows the quantitative expression levels of VEGF, PDG, and ANG-1. An unexpected result was that variant 1/(5.4) appeared to cause a greater increase in expression of VEGF and ANG-1 than did the predominant full-length isoform. Of note, both of the prolines that facilitate HIF-1α degradation are deleted in variant 1/(5.4).

Second Transfection of HIF-1α mRNA into Primary Dermal Fibroblasts

In FIG. 5, analysis with unpaired Student's t-tests demonstrated that predominant and variant 2 had significantly increased levels of HIF-1α compared to control across all three time points (p<0.05). (FIG. 5A) The same analysis showed significant increases of VEGF (FIG. 5B), PGF (FIG. 5C), and ANGPT1 (FIG. 5D) compared to control across all time points for variants 1 and 2.

Third Transfection of HIF-1α mRNA into Primary Dermal Fibroblasts

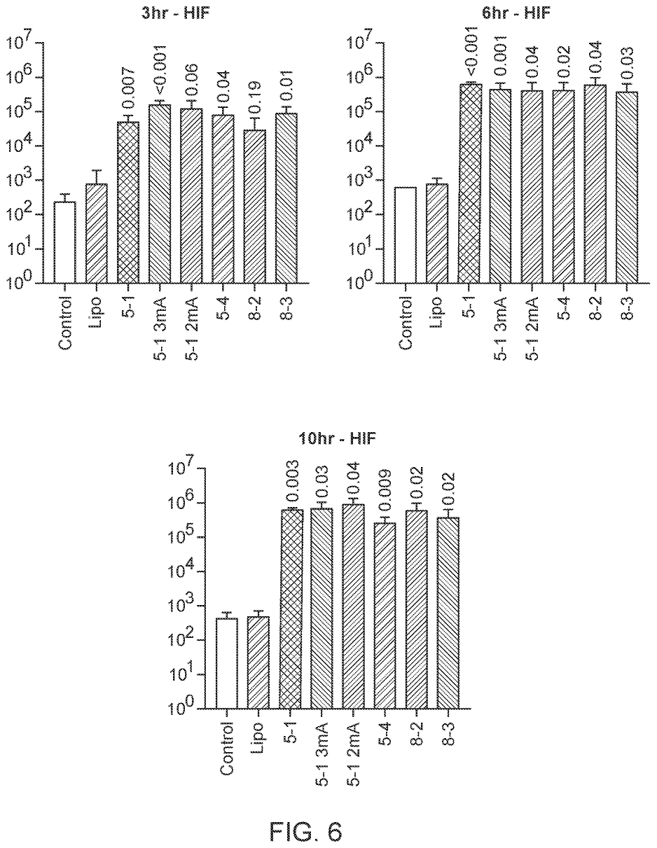

Predominant and three variant HIF-1α 5′-methyl-capped poly-A tailed mRNA were synthesized using T7 RNA polymerase. Primary human fibroblasts were transfected using Lipofectamine MessengerMAX in triplicate, and RNA levels were assessed using RT-qPCR at 3-, 6-, and 10-hour transfection time points. All gene transcript levels were normalized to β-actin expression levels and analyzed by Student's t test. Relative expression levels of HIF-1α, VEGF, PGF, ANGPT1, FLT1, and EDN1 are shown (FIGS. 6, 7, 8, 9, 10, and 11 respectively).

Five different isoforms and mutated isoforms show a significant increase in HIF-1α mRNA levels with a “p” value below 0.05, except for isoform 8-2 at 3 hours post-transfection. At 6 and at 10 hours post-transfection, all of the isoforms and mutated isoforms shows a significant increase in HIF-1α mRNA levels compare with the Lipofectamine only control. (FIG. 6)

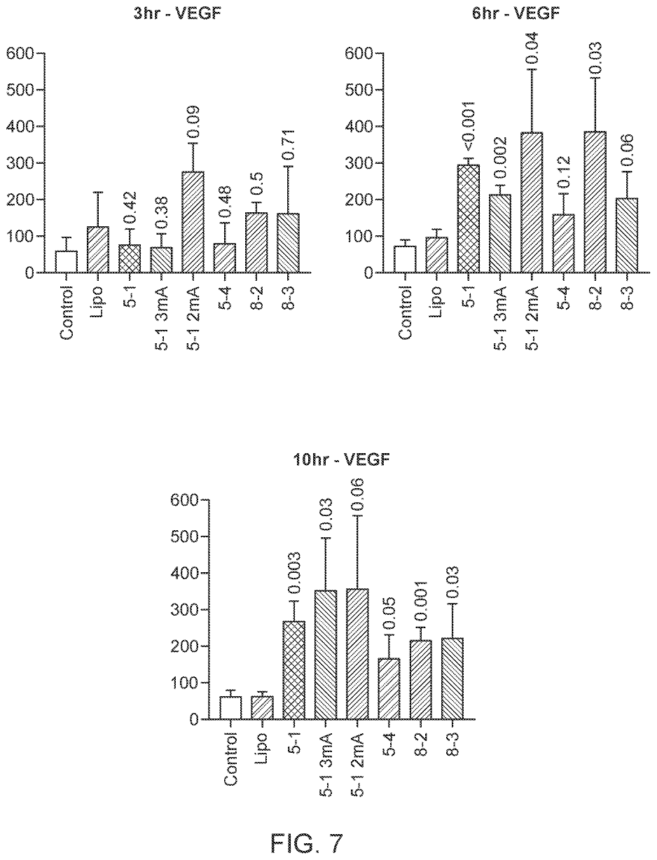

None of the six isoforms and mutated isoforms show a significant difference in VEGF transcript levels with a “p” value below 0.05, at 3 hours post-transfection. At 6 hours post-transfection, all of the isoforms and mutated isoforms show a significant increase in VEGF transcript levels compared with the Lipofectamine only control except for “5-4” and “8-3”. At 10 hours post-transfection, all of the isoforms and mutated isoforms show a significant increase in VEGF transcript levels compared with the Lipofectamine only control except for “5-1 2 mA” and “5-4.” (FIG. 7)

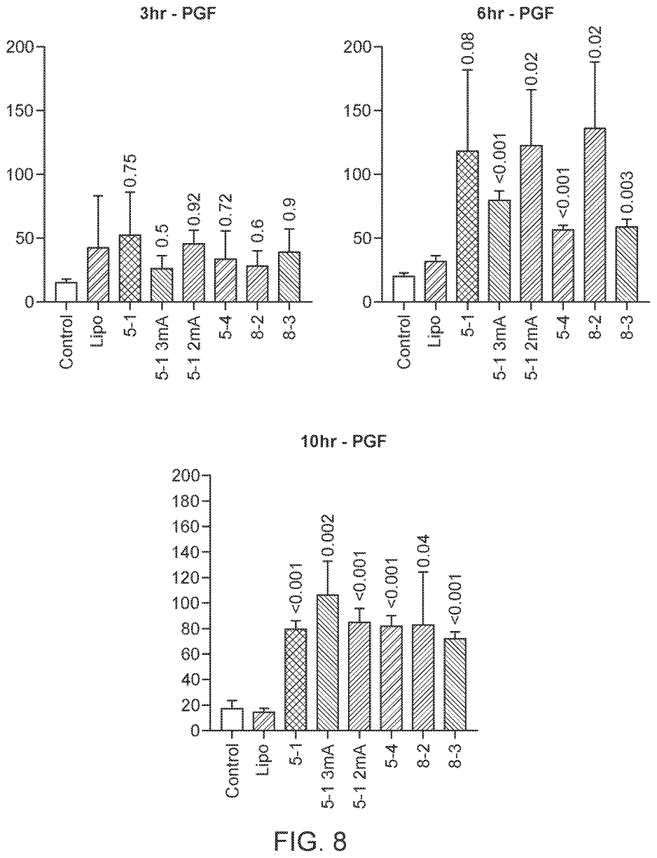

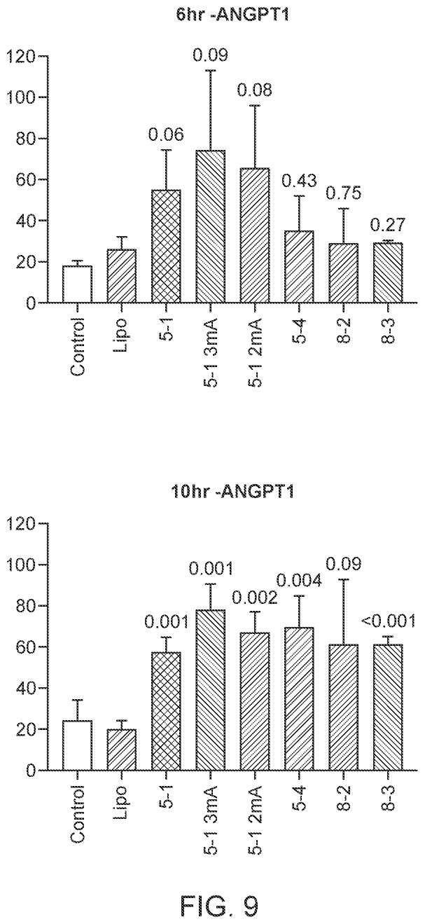

None of the six different isoforms and mutated isoforms show a significant difference in PGF transcript levels with a “p” value below 0.05, at 3 hours post-transfection. At 6 hours post-transfection, all of the isoforms and mutated isoforms show a significant increase in PGF transcript levels compared with the Lipofectamine only control except for “5-1.” At 10 hours post-transfection, all of the isoforms and mutated isoforms show a significant increase in PGF transcript levels compared with the Lipofectamine only control. (FIG. 8)

None of the six different isoforms and mutated isoforms show a significant difference in ANGPT1 transcript levels with a “p” value below 0.05, at 6 hours post-transfection. At 10 hours post-transfection, all of the isoforms and mutated isoforms show a significant increase in ANGPT1 transcript levels compared with the Lipofectamine only control except for “8-2.” (FIG. 9)

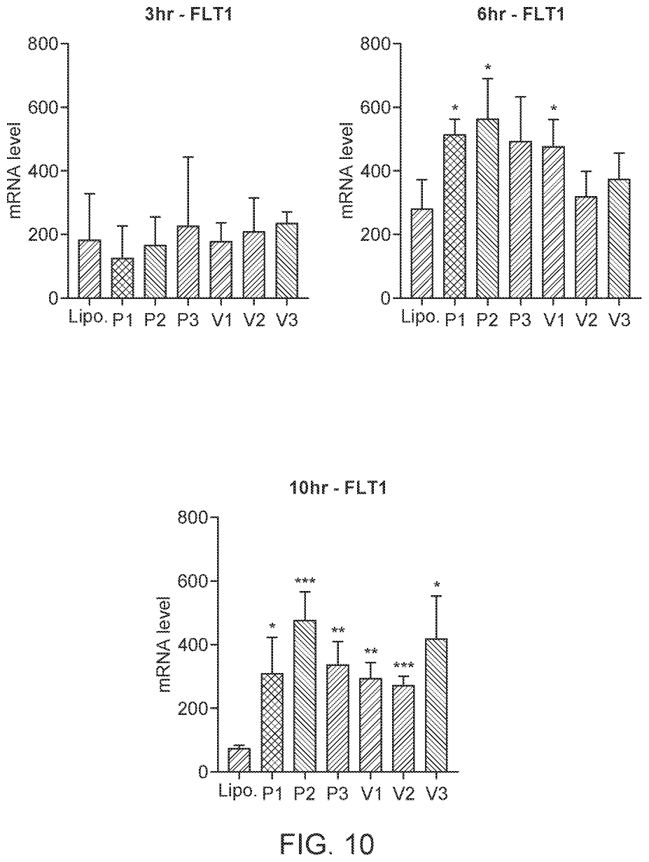

Three (P1, P2 and V1) of the six different isoforms and mutated isoforms show a significant increase in FLT1 transcript levels with a “p” value below 0.05, at 6 hours post-transfection. At 10 hours post-transfection, all of the isoforms and mutated isoforms show a significant increase in FLT1 transcript levels compared with the Lipofectamine only control. (FIG. 10)

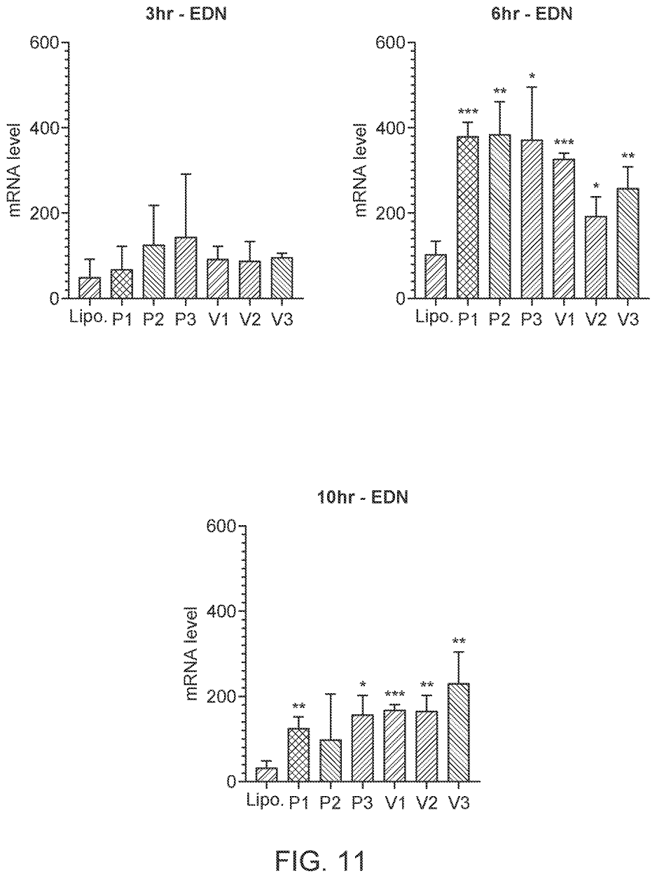

All of the six different isoforms and mutated isoforms show a significant increase in EDN transcript levels with a “p” value below 0.05, at 6 hours post-transfection. At 10 hours post-transfection, five of the isoforms and mutated isoforms show a significant increase in EDN transcript levels compared with the Lipofectamine only control. (FIG. 11)

Notably, constructs P2 and P3 which carry both proline mutations that impair hydroxylation, thus leading to a more stable HIF-1α, can induce an increase in expression of EDN, FLT1, ANGPT1, VEGF, PGF genes.

### Description of the Different HIF-1α Isoforms Shown in FIGS. 6, 7, 8, 9, 10, and 11 (FIG. 12 and Table 1)

“P1,” or (5.1) is the full-length predominant version (826 amino acids) that is shown in NCBI Reference Sequence NM_001530.4 with an exception. At nucleotide 1011, “5-1” has a Guanine instead of the reported Adenosine, which changes the Reference Sequence Lysine to an Arginine in “5-1” (K240R) and P402A and P564A that are involved in HIF-1α degradation via the VHL-mediated ubiquitin protease pathway.

“P2” or (5.1 3 mA), has the mutations K240R, P402A and P564A.

“P3,” or (5.1 2 mA) retains the Arginine at nucleotide 1011, but has the two Prolines changed to alternate amino acids.

“V1,” or (5.4) is a naturally occurring isoform that has a deletion and is only 424 amino acids; Proline at position 402 is intact and has not been mutated.

“V2,” or (8.2) is a naturally occurring isoform that contains 516 amino acids and has only one of the prolines reported to have a role in degradation. Proline at position 402 is intact and has not been mutated.

“V3,” or (8.3) is a naturally occurring isoform that contains 785 amino acids and has both of the Prolines (P402 and P564) reported to have a role in degradation.

### Discussion

Hypoxia-Inducible Factor-1α (HIF-1α) has presented a new direction for ischemic preconditioning of surgical flaps. HIF-1α promotes angiogenesis by upregulating the expression of VEGF, PDGF, angiopoietin, PGF, and other angiogenic proteins. It is therefore attractive to consider the possibility of elevating levels of HIF-1α medically. DNA plasmid delivery for HIF-1α produces a continuing stream of the therapeutic. The HIF-1α molecule may be modified to delete two prolines which increases its resistance to prolyl hydroxylases that function to destroy HIF-1α. Surgical pedicle flap survival in the rat model was improved by pre-treating the flaps with HIF-1α using the DNA plasmid vector. This approach has the advantage of delivering HIF-1a in a localized manner just to the area of interest. It is not systemic. Not surprisingly, other strategies for treating ischemic tissue with HIF-1α are being explored. Approaches for inhibiting prolyl hydroxylases have been utilized, and an inhibitor Dimethyloxalyl-glycine (DMOG) has been applied topically. In another approach, silencing RNA has been applied and has promoted healing and angiogenesis in excisional wounds in diabetic mice. The most extensively assessed HIF-1α stabilizing agent is deferoxamine, and results have been similar to those with the DNA plasmid.

Surgeries, both elective and non-elective, are a major part of world-wide patient medical treatment. An agent/drug that can facilitate faster and more complete wound healing will become a major economic factor in the business of medicine. The potential use of HIF-1α mRNA transcripts is fast, relatively inexpensive, and convenient. Transfection of HIF-1α mRNA into primary dermal fibroblasts results in the rapid, intense, yet transient elevation of HIF-1α response genes which are vital for the establishment and maintenance of a vascular blood supply. The HIF-1α mRNA may be injected or administered into and near the wound-healing site. If additional doses are required, these can be administered when needed. It is very probable that an ointment containing HIF-1α mRNA could also be developed and used to facilitate wound-healing care. Such a treatment could become routine not only for use in surgical patients, but even with relatively minor cuts and bruises.

An additional use and market for this technology could include the veterinary use of HIF-1α for animal wound healing. The vast majority of cats and dogs undergo surgical neutering operation that could find benefit and faster healing following the administration of the HIF-1α drug. The potential market for veterinary use is significant. The use of HIF-1α mRNA to facilitate and improve wound healing in a very large variety of situations is a definite and distinct medical possibility.

1. A synthetic HIF-1α mRNA comprising an open reading frame encoding at least one of a polypeptide derived from a HIF-1α protein or a fragment thereof, wherein the polypeptide or fragment thereof elicits the expression of at least one of an angiogenic factor involved in wound healing.

2. The synthetic HIF-1α mRNA of claim 1, wherein the wound is caused by a surgical procedure and is reconstructed with a pedicle flap.

3. The synthetic HIF-1α mRNA of claim 1, wherein the open reading frame comprises at least one of a mutation encoding for an amino acid residue susceptible to hydroxylation under conditions of normoxia,

4. The synthetic HIF-1α mRNA of claim 3, wherein the amino acid residue is a proline.

5. The synthetic HIF-1α mRNA of claim 4, wherein the amino acid residue is the proline residue at position 564 or the proline residue at position 402, and wherein the numbering of the amino acid residues is that of the human HIF-1α protein.

6. The synthetic HIF-1α mRNA of claim 1, wherein the open reading frame comprises at least one of a nucleic acid sequence having at least 90%, at least 95% or 100% identity with the nucleic acid sequence selected from the group consisting of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, and ID NO: 24, and a fragment thereof.

7. The synthetic HIF-1α mRNA of claim 1, wherein the open reading frame comprises at least one of a nucleic acid sequence having at least 90%, at least 95% or 100% identity with the nucleic acid sequence SEQ ID NO: 17 and a fragment thereof.

8. The synthetic HIF-1α mRNA of claim 1, wherein the open reading frame comprises at least one of a nucleic acid sequence having at least 90%, at least 95% or 100% identity with the nucleic acid sequence SEQ ID NO: 18 and a fragment thereof.

9. The synthetic HIF-1α mRNA of claim 1, further comprising at least one of a poly-A tail, a methylated cap, a 5′ untranslated region (5′-UTR), a 3′ untranslated region (3′-UTR) or any combination thereof.

10. The synthetic HIF-1α mRNA of claim 1, wherein at least one of a nucleobase of the nucleic acid sequence is modified by methylation, isomerization or both.

11. The synthetic HIF-1α mRNA of claim 1, wherein the at least one of the nucleobase is selected from the group consisting of pseudouridine (PsU), N1-methyl-pseudouridine (1mPsU), 5-methyl-uridine (5mU), 2′-O-methyl-uridine (2′-O-methyl-U), 5-methyl-cytidine (5mC), 2-thiouridine (s2U), N6-methyladenosine (6 mA), and any combination thereof.

12. The synthetic HIF-1α mRNA of claim 11, wherein the at least one of the nucleobase is N1-methyl-pseudouridine (1mPsU).

13. The synthetic HIF-1α mRNA of claim 1, wherein the at least one of the angiogenic factors is selected from the group of consisting of vascular endothelial growth factor (VEGF), placental growth factor (PGF), angiopoietin 1 (ANGPT1), angiopoietin 2 (ANGPT2), erythropoietin (EPO), vascular endothelial growth factor receptor 1 (VEGFR1), fms-related receptor tyrosine kinase 1 (FLT1), and endothelin (EDN1).

14. A therapeutic composition comprising at least one of the synthetic HIF-1α mRNA of claim 1.

15. The therapeutic composition of claim 14, further comprising at least one of a pharmaceutically acceptable carrier.

16. The therapeutic composition of claim 15, wherein the at least one of the synthetic HIF-1α mRNA is complexed or associated with at least one of a cationic lipid or an ionizable lipid.

17. The therapeutic composition of claim 16, wherein the at least one of the cationic lipid or the ionizable lipid form a lipid-based carrier selected from the group consisting of a liposome and a lipid nanoparticle (LNP).

18. The therapeutic composition of claim 17, wherein the lipid-based carrier additionally comprises at least one of a neutral lipid, a steroid or steroid analogue, or any combination thereof.

19. The therapeutic composition of claim 17, wherein the lipid-based carrier is a liposome.

20. The therapeutic composition of claim 19, wherein the liposome is lipofectamine.

21. The therapeutic composition of claim 17, wherein the LNP comprises a PEGylated lipid.

22. The therapeutic composition of claim 14, wherein the at least one of the synthetic HIF-1α mRNA is complexed with at least one histidine-lysine co-polymer (HKP) to form a polymeric nanoparticle.

23. The therapeutic composition of claim 22, wherein the polymeric nanoparticle further comprises at least one ionizable lipid.

24. A Kit comprising the synthetized HIF-1α mRNA of claim 1.

25. The kit of claim 24, further comprising technical instructions providing information on administration and dosage of the synthetized HIF-1α mRNA or therapeutic composition.

26. The kit of claim 24, wherein the synthetized HIF-1α mRNA or therapeutic composition is lyophilized, and wherein the kit further comprises a solubilizing liquid.

27. A method for producing the synthetic HIF-1α mRNA of claim 1 comprising:
providing a complementary deoxyribonucleic acid (cDNA) encoding at least one of a polypeptide derived from a HIF-1α protein or a fragment thereof, and
contacting the provided cDNA with an RNA polymerase in the presence of nucleotides, under conditions such that the mRNA is synthesized.

28. The method of claim 27, wherein the RNA polymerase is a T7 RNA polymerase.

29. The method of claim 27, wherein the nucleobase of at least one of the nucleotide is modified by methylation, isomerization or both.

30. A method for treating a surgical wound or preventing ischemia or necrosis in wound tissue, comprising administering to a subject in need an effective amount of the synthetic HIF-1α mRNA of claim 1.

31. The method of claim 30, wherein the wound is reconstructed with a pedicle flap.

32. The method of claim 30, wherein the administering of the synthetic HIF-1α mRNA or the therapeutic composition causes a HIF-1-induced response.

31. The method of claim 30, wherein the HIF-1-induced response promotes the neovascularization and/or maintains the vascularization of the distal portion of the flap.

34. The method of claim 31, wherein the wound is from a reconstruction after traumatic injury, complex abdominal hernia repair, breast reconstruction after resection for cancer, and plastic surgical procedures including abdominoplasties and facelifts.

35. The method of claim 30, wherein the synthetic HIF-1α mRNA or the therapeutic composition is administered subcutaneously, intramuscularly, or transdermally.

36. The method of claim 30, wherein the subject is a human being or an animal.

37. The method of claim 36, wherein the human being has a medical condition that increases the risk of ischemia or necrosis during wound healing.

38. The method of claim 36, wherein the animal is a cat or a dog and has undergone surgical sterilization.

39. The synthetic HIF-1α mRNA of claim 1.

40. The synthetic HIF-1α mRNA or the therapeutic composition for use according to claim 39, wherein the surgical wound is reconstructed with a pedicle flap.

41. The synthetic HIF-1α mRNA or the therapeutic composition for use according to claim 40, wherein the pedicle flap is at risk of ischemia or necrosis.


SEQUENCE LISTING (24 sequences)

SEQ ID NO: 1 - DNA, 3946 bp, synthetic construct
  source 1..3946: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaggactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 2 - DNA, 3946 bp, synthetic construct
  source 1..3946: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccgcagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctgcctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 3 - DNA, 3946 bp, synthetic construct
  source 1..3946: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaggactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccgcagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctgcctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 4 - DNA, 3102 bp, synthetic construct
  source 1..3102: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactacacaggccacattcacgtatatgataccaacagtaaccaacctcagtgtgggtataagaaaccacctgtgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggatcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 5 - DNA, 3389 bp, synthetic construct
  source 1..3389: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacaggatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 6 - DNA, 3823 bp, synthetic construct
  source 1..3823: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacgatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgctgaatgctcagagaaagcgaaaaatggaacatgatggttcactttttcaagcagtaggaattggaacattattacagcagccagacgatcatgcagctactacatcactttcttggaaacgtgtagaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 7 - DNA, 3946 bp, Homo sapiens
  source 1..3946: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 8 - DNA, 3819 bp, Homo sapiens
  source 1..3819: mol_type = other DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 9 - DNA, 3865 bp, Homo sapiens
  source 1..3865: mol_type = other DNA
aataagtggtggttactcagcacttttagatgctgtttataatagatgaccttttctaactaatttacagttttttgaaagataactgagaggttgagggacggagattttcttcaagcaattttttttttcattttaaatgagctcccaatgtcggagtttggaaaacaaatttgtctttttaaaagaaggtctaggaaactcaaaacctgaagaattggaagaaatcagaatagaaaatggtaggataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 10 - protein, 825 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSRTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 11 - protein, 826 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAAAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAAYIPMDDDFQLRSFDQLSPLESSSASPESASPQSTVTVFQQTQIQEPTANATTTTATTDELKTVTKDRMEDIKILIASPSPTHIHKETTSATSSPYRDTRSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 12 - protein, 826 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSRTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAAAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAAYIPMDDDFQLRSFDQLSPLESSSASPESASPQSTVTVFQQTQIQEPTANATTTTATTDELKTVTKDRMEDIKILIASPSPTHIHKETTSATSSPYRDTRSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 13 - protein, 424 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHYTGHIHVYDTNSNQPQCGYKKPPVTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNELQFLRKN

SEQ ID NO: 14 - protein, 516 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTGCVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPENYSS

SEQ ID NO: 15 - protein, 785 aa, synthetic construct
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALLNAQRKRKMEHDGSLFQAVGIGTLLQQPDDHAATTSLSWKRVEGCKSSEQNGMEQKTIILIPSDLACRLLGQSMDESGLPQLTSYDCEVNAPIQGSRNLLQGEELLRALDQVN

SEQ ID NO: 16 - RNA, 3946 nt, synthetic construct
  source 1..3946: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaggactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 17 - RNA, 3946 nt, synthetic construct
  source 1..3946: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccgcagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctgcctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 18 - RNA, 3946 nt, synthetic construct
  source 1..3946: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaggactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccgcagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctgcctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcgaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 19 - RNA, 3102 nt, synthetic construct
  source 1..3102: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactacacaggccacattcacgtatatgataccaacagtaaccaacctcagtgtgggtataagaaaccacctgtgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggatcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 20 - RNA, 3389 nt, synthetic construct
  source 1..3389: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacaggatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 21 - RNA, 3823 nt, synthetic construct
  source 1..3823: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacgatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgctgaatgctcagagaaagcgaaaaatggaacatgatggttcactttttcaagcagtaggaattggaacattattacagcagccagacgatcatgcagctactacatcactttcttggaaacgtgtagaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 22 - RNA, 3946 nt, Homo sapiens
  source 1..3946: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 23 - RNA, 3819 nt, Homo sapiens
  source 1..3819: mol_type = other RNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 24 - RNA, 3865 nt, Homo sapiens
  source 1..3865: mol_type = other RNA
aataagtggtggttactcagcacttttagatgctgtttataatagatgaccttttctaactaatttacagttttttgaaagataactgagaggttgagggacggagattttcttcaagcaattttttttttcattttaaatgagctcccaatgtcggagtttggaaaacaaatttgtctttttaaaagaaggtctaggaaactcaaaacctgaagaattggaagaaatcagaatagaaaatggtaggataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg